(19) United States
(12) Patent Application Publication
(10) Pub. No.: US 2005/0182397 A1
(43) Pub. Date: Aug. 18, 2005

(54) Device and method for ablation of body cavities

(76) Inventor: Thomas Ryan, Flemington, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/771,765

(22) Filed: Feb. 4, 2004

(51) Int. Cl. A61B 18/04

(52) U.S. Cl. 606/28; 606/29

(57) ABSTRACT

A device for ablating a body cavity and a method for using the same. The device includes an introducer having a distal end and a proximal end and at least one channel therethrough, a distendable bladder coupled to the distal end and being distendable within the body cavity from a substantially deflated state to an inflated state wherein it approximates an interior of at least a portion of said body cavity that is to be ablated, and an inflation device coupled to the proximal end and in fluid communication with the at least one channel and with an interior of the distendable bladder. Activation of the inflation device causes an inflation medium to flow through the at least one channel and into the distendable bladder to thereby inflate the distendable bladder. The device also includes at least one flexible resistive element coupled to the distendable bladder. The resistive element is electrically coupleable to a voltage source and emits resistive heat when so coupled. The resistive element is coupled to the distendable bladder in a manner so as not to impair movement of the bladder from the deflated to the inflated states.

### BACKGROUND OF THE INVENTION

1. Field of the Invention

The present invention relates generally to devices and methods for ablating body cavities, and more particularly to an expandable device for body cavity ablation that utilizes one or more resistive heating elements to cause or assist in such ablation.

2. Background Discussion

Removal of the uterine endometrium has proven an excellent alternative to a full hysterectomy in the surgical treatment of abnormal uterine bleeding, a symptom of menorraghia. A variety of devices and associated techniques for removal of the endometrium are well known, and include endometrial resection, ablation by laser treatment or electrosurgery, and thermal or cryogenic cauterization of the endometrium.

With regard to thermal cauterization or ablation of the endometrium, one known device and associated technique involves heating fluid within an expandable fluid filled balloon until ablation is achieved, as is described in more detail in U.S. Pat. No. 4,949,718, which is incorporated herein by reference. One challenge of such devices is to achieve the maximum amount of coverage possible. In other words, it is desirable to achieve sufficient contact between the expandable balloon and the endometrial lining so that 100% ablation is achieved. This has not always been possible, however, as the shape of the uterus, in combination with the fact that there often are fibroids present, render it difficult to get uniform contact throughout the entire uterine interior. Another challenge is to achieve uniform surface heating to ensure adequate and uniform ablation. With the use of a fluid filled balloon, some variations in fluid temperature, particularly in difficult to reach areas such as the corneal areas can occur. Further, given the fact that the objective with fluid filled balloons is to provide uniform heating, no means is provided for targeted heating, such as in the corneal areas or the fundus areas to overcome the above-described difficulties. Some known devices, such as that described in U.S. Pat. No. 5,443,470, have incorporated multiple radiofrequency (RF) electrodes on the surface of a balloon. RF electrodes, however, can only provide effective heating and ablation when there is direct tissue contact, as such contact is necessary to complete the electrical path. Thus, these devices are prone to the same problems and challenges in achieving adequate coverage.

Accordingly, there is a need for an improved device and method for ablating body cavities, and for such a device and method having particular application for endometrial ablation.

### SUMMARY OF THE INVENTION

A device for ablating a body cavity is provided including an introducer having a distal end and a proximal end and at least one channel therethrough, a distendable bladder coupled to the distal end and being distendable within the body cavity from a substantially deflated state to an inflated state wherein it approximates an interior of at least a portion of said body cavity that is to be ablated, and an inflation device coupled to the proximal end and in fluid communication with the at least one channel and with an interior of the distendable bladder. Activation of the inflation device causes an inflation medium to flow through the at least one channel and into the distendable bladder to thereby inflate the distendable bladder. The device further includes at least one flexible resistive element coupled to the distendable bladder. The resistive element is electrically coupleable to a voltage source and emits resistive heat when so coupled. It is further coupled to the distendable bladder in a manner so as not to impair movement of the bladder from the deflated to the inflated states.

In alternate embodiments, the at least one resistive element is coupled to either an inner surface or an outer surface of the distendable bladder. In one embodiment, the at least one resistive element substantially covers a surface area of the distendable bladder, and in yet another embodiment, the resistive element is coupled to the distendable bladder along a serpentine path so as to cover a predetermined portion of a surface area of the bladder. In yet another embodiment, the device further includes a plurality of flexible resistive elements.

According to yet another embodiment, each of the plurality of flexible resistive elements are coupled to the distendable bladder along a serpentine path so as to cover respective predetermined portions of the surface area of the bladder, and in a further embodiment, each of the plurality of flexible resistive elements are separately coupleable to a separate voltage source.

In one embodiment, the body cavity is the uterus, and, when in the inflated state, the distendable bladder approximates an interior of the uterus. In a further embodiment, each of the first and second flexible resistive elements are coupled to the distendable bladder along a serpentine path so as to cover predetermined first and second portions of the surface area of the bladder respectively, and when the distendable bladder is in the inflated state, the first and second resistive elements are in thermal contact with first and second portions of the endometrial lining of the uterus. In yet a further embodiment, the first and second portions of the endometrial lining are in first and second corneal areas of the uterus.

In yet further alternate embodiments, the inflation medium is a fluid, a gas, or specifically air.

Also provided is a device for ablating a body cavity including an introducer having a distal end and a proximal end and at least one channel therethrough, an expandable element coupled to the distal end and being expandable within the body cavity from a substantially collapsed state to an expanded state wherein its configuration approximates an interior of said body cavity, an expansion mechanism coupled to the proximal end of the introducer for moving the expandable structure between the collapsed and expanded states, and at least one flexible resistive element coupled to the expandable structure. The resistive element is electrically coupleable to a voltage source and emits resistive heat when so coupled. The resistive element is further coupled to the expandable structure so as to move therewith between the collapsed and expanded states.

In one embodiment, the device further includes a plurality of flexible resistive elements each of which is individually coupleable to a separate voltage source.

In yet another embodiment, the body cavity is the uterus, and when in the expanded state, the expandable structure has a configuration that approximates an interior of the uterus. In a further embodiment, when in the expanded state, the at least one resistive element is in thermal contact with a substantial portion of the endometrial lining of the uterus, and in yet another embodiment, when in the expanded state, the at least one resistive element is in thermal contact with a portion of the endometrial lining of the uterus.

A method is also provided for ablating a body cavity. The method includes providing an introducer having a distal end and a proximal end, an expandable element coupled to the distal end and being expandable within the body cavity from a substantially collapsed state to an expanded state wherein it approximates an interior of a patient's uterus, an expansion device the activation of which causes the expandable element to move between the collapsed and expanded states, and at least one flexible resistive element coupled to the expandable element for movement therewith and electrically coupleable to a voltage source and emitting resistive heat when so coupled. The method further includes, with the expandable element in the retracted state, inserting the device into the patient's uterus so that the expandable element is positioned within the uterus, activating the expansion device so that the expandable element moves from the collapsed to the expanded state, coupling the at least one resistive element to a voltage source so as to cause it to emit resistive heat, and heating a portion of the endometrial lining of the uterus that is in thermal contact with the resistive element to thereby cause tissue necrosis.

In one embodiment, the expandable element is a distendable bladder, and in yet another embodiment, the expansion device is a device for injecting an inflation medium into the distendable bladder. In yet another embodiment, the device for injecting is a syringe.

Finally, a device is also provided for ablating a uterus. The device includes an introducer having a distal end and a proximal end and at least one channel therethrough, a distendable bladder coupled to the distal end and being distendable within the body cavity from a substantially deflated state to an inflated state wherein it approximates an interior of at least a portion of the uterus, an inflation device coupled to the proximal end and in fluid communication with the at least one channel and with an interior of the distendable bladder. Activation of the inflation device causes an inflation medium to flow through the at least one channel and into the distendable bladder to thereby inflate the distendable bladder. The device further includes at least a first long, thin flexible element coupled to the distendable bladder in the region of the bladder in proximity to a first corneal area of the uterus when the bladder is positioned within the uterus, and at least a second long, thin flexible element coupled to the distendable bladder in the region of the bladder in proximity to a second corneal area of the uterus when the bladder is positioned within the uterus. The at least first and second flexible elements are coupled to the distendable bladder in a manner so as to direct expansion of the bladder into the corneal regions of the uterus.

In one embodiment, the first and second flexible elements are arranged to form a pattern of substantially concentric circles, with the center of said substantially concentric circles being substantially aligned in the direction of the center of the respective corneal regions.

These and other features and advantages of the present invention will become apparent from the following more detailed description, when taken in conjunction with the accompanying drawings which illustrate, by way of example, the principles of the invention.

### BRIEF DESCRIPTION OF THE DRAWINGS

FIG. 1 illustrates a device according to the present invention in an inflated state within a body cavity;

FIG. 2 illustrates the device of FIG. 1 having a different configuration for the resistive elements and in a deflated state;

FIG. 3 illustrates the device of FIG. 1 coupled to an inflation device and a control unit;

FIG. 3a illustrates a portion of the interior of the control unit of FIG. 3;

FIG. 4 illustrates an alternate embodiment of the present invention having resistive elements coupled to predetermined portions of the distendable bladder;

FIG. 5 illustrates yet another embodiment of the present invention having multiple resistive elements;

FIG. 5a is an enlarged view of a portion of FIG. 5, showing additional temperature sensors; and

FIGS. 6a-6b illustrate various configurations for resistive elements(s).

### DETAILED DESCRIPTION OF THE INVENTION

Before explaining the present invention in detail, it should be noted that the invention is not limited in its application or use to the details of construction and arrangement of parts illustrated in the accompanying drawings and description. The illustrative embodiments of the invention may be implemented or incorporated in other embodiments, and variations and modifications thereof may be practiced or carried out in various ways.

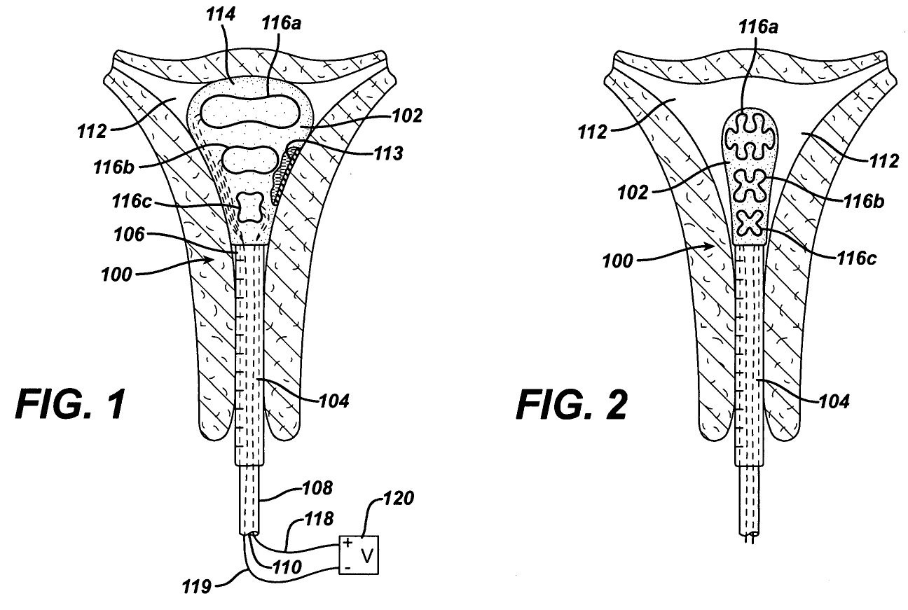

According to one embodiment of the present invention shown in FIGS. 1-3, a device 100 for ablating a body cavity 112, such as the uterus, includes an introducer 104, such as a catheter or other tube-like element, having a distal end 106, a proximal end 108 and at least one channel 110 extending through it. A distendable bladder 102 is secured to the distal end of the introducer. The distendable bladder is distendable from a substantially deflated state shown in FIG. 2, to a substantially inflated state shown in FIG. 1. The distendable bladder is designed such that, in the deflated state, it is of a sufficiently small configuration (as is the introducer) so that it can be inserted into the body cavity 112, the surface or lining of which is to be ablated. Once inserted, the distendable bladder is designed such that, when in the inflated state, the distendable bladder substantially approximates the interior of the body cavity, or at least that portion of the body cavity that is to be ablated. The distendable bladder further has an inner surface 113 and an outer surface 114.

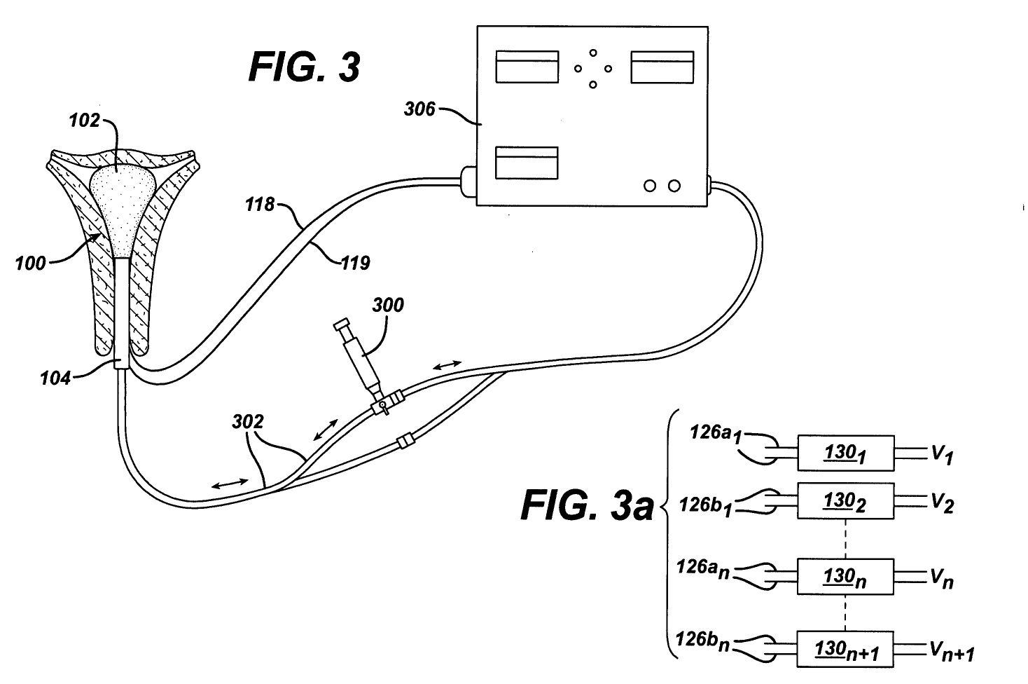

The interior of the distendable bladder is in fluid communication with the channel 110 extending through the introducer 104. The proximal end 108 of the channel 110 is further coupled to an inflation device capable of causing an inflation medium to flow through the channel and into the interior of the distendable bladder to cause the distendable bladder to assume its inflated configuration. One such inflation device is illustrated in FIG. 3, where a syringe 300 or the like is coupled to tubing 302 that is coupled to the proximal end of the introducer. The syringe may be used to inject any suitable inflation medium into the balloon, such as water, saline, or air. A sufficient amount of such inflation medium should be injected into the distendable bladder to maintain the pressure within the bladder at approximately 40-140 mmHg, preferably 75 mm Hg, as excessive pressures can lead to possibly internal injury of the patient. Although a syringe type mechanism is illustrated in FIG. 3, those skilled in the art will recognize that any suitable means for injecting an inflation medium into the distendable bladder can be used.

The distendable bladder must be capable of withstanding a significant amount of heat applied at its surface, as will be described further below, without rupturing or otherwise degrading. Suitable materials for the distendable bladder include silicone, latex, polyurethane and polyvinylchloride, preferably with a thickness between 0.07 and 0.127 mm.

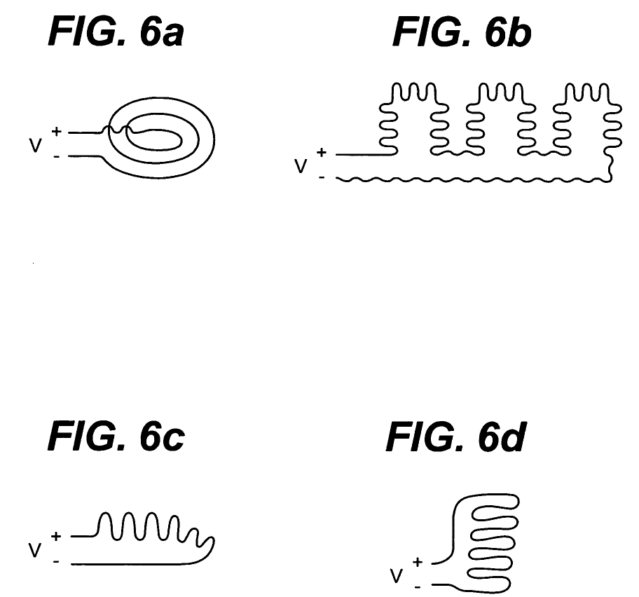

Returning now to FIGS. 1 and 2, the device further includes at least one flexible resistive element coupled to the distendable bladder. In the embodiment illustrated in FIG. 1, multiple resistive elements 116a, 116b, 116c are positioned at desired locations about the outer surface of the distendable bladder. Although three resistive elements are shown on one side of the embodiment of FIG. 1, this is for illustrative purposes only and such resistive elements would likely be present around the circumference of the distendable bladder. It is to be understood that any number of resistive elements could be used, and that they could be placed at any desired location on the outer surface of the balloon. The number and placement of resistive elements should be sufficient to obtain the desired coverage. For example, multiple resistive elements similar to those shown in FIG. 1 or 2 could be spaced at substantially uniform distances so as to cover substantially the entire outer surface of the distendable bladder. Further, various configurations for the pattern of the resistive elements are also possible, such as those shown in FIGS. 6a-6d. Although many suitable configurations are conceivable, the configurations must be such that the resistive elements allow the desired expansion of the distendable bladder in the desired directions and at the desired locations.

Returning now to FIG. 1, leads 118, 119 are electrically coupled to the resistive elements and extend from the bladder through or along the introducer to a voltage source 120 positioned external to the patient. This voltage source may be contained within a control unit 306 such as that shown in FIG. 3. In addition, as will be described further below, the control unit may display temperature and/or pressure readings from thermocouples, pressure transducers or the like that are coupled to or positioned within the distendable bladder. Leads 118, 119 from the resistive element (and any transducers or thermocouples) may extend through the same channel described above, or may extend through a separate channel through the introducer. Preferably, the resistive element leads are connected to copper wires that supply voltage without corresponding heating. Although the flexible resistive element is shown on the outside surface of the distendable bladder, it may also be coupled to its interior surface, or be contained within the balloon wall.

The flexible resistive element is simply a thin gauge wire that emits resistive heat when the voltage from voltage source 120 is applied. In one embodiment, the resistive elements are nickel-based alloy resistance wires, such as nickel-chrome wire or nickel-copper alloys, having a heating range between 0.1 and 1.0 watts/cm². The element should further be flexible enough so that it does not impair movement of the bladder from the deflated to inflated states. To achieve this, the resistive element is preferably coupled to the bladder by direct gluing or overlay of a material with the resistive elements already embedded. One way to achieve this is by dipping a balloon shaped mandrel into a solution of silicon and toluene or other suitable solvent. The dilution ratio of the silicon solution determines the thickness added to the balloon with each dip. Preferably, each dip will add approximately 0.001 inches in thickness, and the balloon is dipped two times. Following the second dip, the resistive elements are laid over the balloon in the desired pattern, and the balloon then dipped a third and fourth time to cover the resistive elements. It is also possible, after one or two initial dips, to spray a silicone adhesive over the balloon before laying the pattern of the resistive elements over the balloon. In this case, it may be optional whether to subsequently dip the balloon again.

In an alternate method, the shape of the mandrel is that of the final, distended state of the distendable bladder. Following dipping and laying of the resistive elements as described above, the balloon is rolled into a cylinder for packaging prior to sterilization.

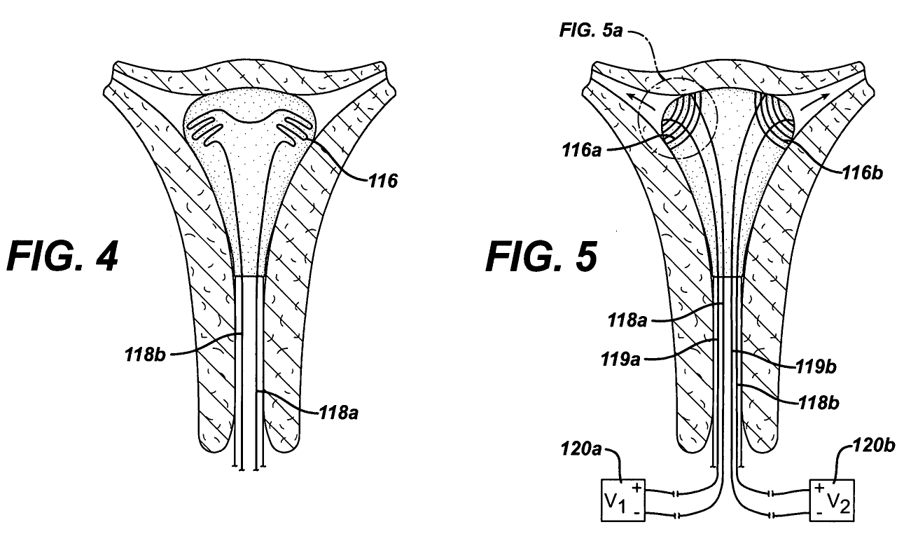

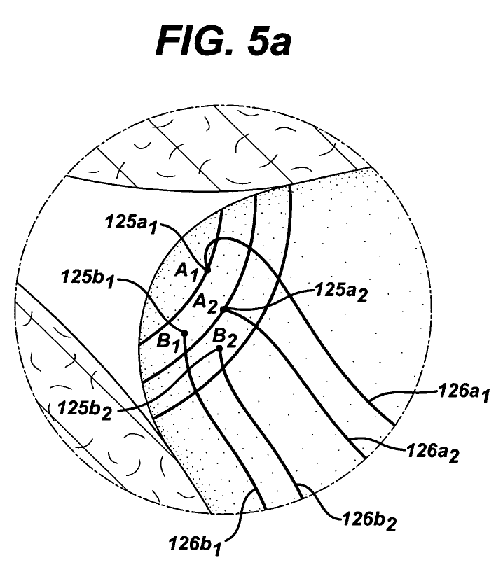

In yet another alternate method, the balloon is coated with a thin layer of metal, preferably using vapor deposition techniques. The desired paths of the resistive elements are then coated with an acrylic or similar insulating coating, and the balloon etched in acid in a manner similar to a printed circuit board, leaving only the desired resistive element paths. FIG. 4 shows an alternative embodiment of the present invention in which a single resistive element 116 may be coupled to the surface of the bladder in such a way that it covers a select portion or portions of the surface of the bladder. In this manner, a hot fluid filled balloon such as that described in U.S. Pat. No. 4,949,718, can be enhanced to enable targeted areas of additional heating, such as may be desirable to achieve better coverage in the corneal areas. As an alternative, two or more flexible resistive elements can be used that can either be coupled to a single voltage source or separably coupled to individual voltage sources. FIG. 5 illustrates such a device wherein first and second flexible resistive elements 116a, 116b are coupled by separate leads 118a, 119a; 118b, 119b to separate voltage sources 120a, 120b. This enables controlled and targeted heating and/or additional heating of one or more areas. It is to be understood that any variation of the number and configuration of resistive elements is possible.

It is noted that the embodiment of FIG. 5 will achieve increased coverage of the corneal areas of the uterus regardless of whether elements 116a, 116b are resistive elements. For example, if any long, thin, flexible element, whether electrically conductive or not, is applied in the illustrated pattern, when the distendable bladder is inflated the elements will restrict expansion of the bladder in the corner regions other than in the direction indicted by the arrows in FIG. 5, which is into the center of the corneal regions of the uterus. Thus, application of elements 116a, 116b alone can improve the coverage achieved by the distendable bladder.

As indicated above, the control unit 306 may display temperature or pressure readings from thermocouples, pressure transducers or the like that are coupled to or positioned within the distendable bladder. FIG. 5a shows an enlarged view of a portion of FIG. 5 in which thermocouples or temperature sensors 125a, 125b are also coupled to the distendable bladder. In the illustrated embodiment, temperature sensors 125a are positioned substantially on the resistive elements whereas temperature sensors 125b are positioned between the resistive elements. Leads 126a, 126b are each a pair of wires that preferably are electrically coupled to separate controller channels within the control unit 306, such as is illustrated schematically in FIG. 3a. Each controller 130 receives input from temperature sensors (i.e., sensors 125a and 125b via wire pairs 126a and 126b respectively), evaluates the input temperatures and compares them to target temperatures for each sensor. If above the target temperature, the voltage V is lowered and the temperatures re-read. Thus, voltage for each segment of the resistive element(s) can be controlled by a such a feed-back loop.

As referred to above, the use of flexible resistive elements coupled to a distendable bladder has many advantages. First, such elements can be used to enable additional controlled heating in targeted locations, such as the cornua or fundus areas, thereby achieving enhanced coverage and/or enhanced depth of penetration in those areas. In the alternative, such resistive elements can provide ablation heating in and of themselves, i.e., without requiring a heated fluid or other medium within the interior of the balloon. Thus, the difficulties in managing the fluid, controlling the temperature of the fluid, and potential risk of fluid leakage can be avoided entirely. Further, there are no limitations on the amount of heat that can be applied, whereas fluid heating is constrained by the boiling point of the fluid. In addition, the use of resistive heating elements as opposed to other known heating elements, such as electrodes, is advantageous in that no direct contact with tissue is required for such devices to be effective.

Finally, the embodiments described above illustrate the use of flexible resistive heating elements in conjunction with an inflatable, distendable bladder. Resistive heating elements may also be used with any other type of shell or other structure that expands to approximate the interior of the cavity to be ablated. For example, flexible resistive elements can be secured to any type of cage or mesh cage structure that is mechanically expandable once inserted into the body cavity.

It will be apparent from the foregoing that, while particular forms of the invention have been illustrated and described, various modifications can be made without departing from the spirit and scope of the invention. Accordingly, it is not intended that the invention be limited, except as by the appended claims.

1. A device for ablating a body cavity comprising:
an introducer having a distal end and a proximal end and at least one channel therethrough;
a distendable bladder coupled to the distal end and being distendable within the body cavity from a substantially deflated state to an inflated state wherein it approximates an interior of at least a portion of said body cavity that is to be ablated;
an inflation device coupled to the proximal end and in fluid communication with the at least one channel and with an interior of the distendable bladder, wherein activation of the inflation device causes an inflation medium to flow through the at least one channel and into the distendable bladder to thereby inflate the distendable bladder; and
at least one flexible resistive element coupled to the distendable bladder, the resistive element being electrically coupleable to a voltage source and emitting resistive heat when so coupled, the resistive element being coupled to the distendable bladder in a manner so as not to impair movement of the bladder from the deflated to the inflated states.

2. The device according to claim 1, wherein the at least one resistive element is coupled to an inner surface of the distendable bladder.

3. The device according to claim 1, wherein the at least one resistive element is coupled to an outer surface of the distendable bladder.

4. The device according to claim 1, wherein the at least one resistive element substantially covers a surface area of the distendable bladder.

5. The device according to claim 1, wherein the resistive element is coupled to the distendable bladder along a serpentine path so as to cover a predetermined portion of a surface area of the bladder.

6. The device according to claim 1, having a plurality of flexible resistive elements.

7. The device according to claim 6, wherein each of the plurality of flexible resistive elements are coupled to the distendable bladder along a serpentine path so as to cover respective predetermined portions of the surface area of the bladder.

8. The device according to claim 6, wherein each of the plurality of flexible resistive elements are separately coupleable to a separate voltage source.

9. The device according to claim 1, wherein the body cavity is the uterus, and wherein, when in the inflated state, the distendable bladder approximates an interior of the uterus.

10. The device according to claim 9, having first and second flexible resistive elements, wherein each of the first and second flexible resistive elements are coupled to the distendable bladder along a serpentine path so as to cover predetermined first and second portions of the surface area of the bladder respectively, and wherein when the distendable bladder is in the inflated state, the first and second resistive elements are in thermal contact with first and second portions of the endometrial lining of the uterus.

11. The device according to claim 10, wherein the first and second portions of the endometrial lining are in first and second corneal areas of the uterus.

12. The device according to claim 1, wherein the inflation medium is a fluid.

13. The device according to claim 1, wherein the inflation medium is a gas.

14. The device according to claim 13, wherein the inflation medium is air.

15. A device for ablating a body cavity comprising:
an introducer having a distal end and a proximal end and at least one channel therethrough;
an expandable element coupled to the distal end and being expandable within the body cavity from a substantially collapsed state to an expanded state wherein its configuration approximates an interior of said body cavity;
an expansion mechanism coupled to the proximal end of the introducer for moving the expandable structure between the collapsed and expanded states; and
at least one flexible resistive element coupled to the expandable structure, the resistive element being electrically coupleable to a voltage source and emitting resistive heat when so coupled, the resistive element being coupled to the expandable structure so as to move therewith between the collapsed and expanded states.

16. The device according to claim 15, having a plurality of flexible resistive elements, each one being individually coupleable to a separate voltage source.

17. The device according to claim 15, wherein the body cavity is the uterus, and wherein, when in the expanded state, the expandable structure has a configuration that approximates an interior of the uterus.

18. The device according to claim 15, wherein when in the expanded state, the at least one resistive element is in thermal contact with a substantial portion of the endometrial lining of the uterus.

19. The device according to claim 15, wherein when in the expanded state, the at least one resistive element is in thermal contact with a portion of the endometrial lining of the uterus.

20. A method for ablating a body cavity, comprising the steps of:
providing an introducer having a distal end and a proximal end, an expandable element coupled to the distal end and being expandable within the body cavity from a substantially collapsed state to an expanded state wherein it approximates an interior of a patient's uterus, an expansion device the activation of which causes the expandable element to move between the collapsed and expanded states, and at least one flexible resistive element coupled to the expandable element for movement therewith and electrically coupleable to a voltage source and emitting resistive heat when so coupled;
with the expandable element in the retracted state, inserting the device into the patient's uterus so that the expandable element is positioned within the uterus;
activating the expansion device so that the expandable element moves from the collapsed to the expanded state;
coupling the at least one resistive element to a voltage source so as to cause it to emit resistive heat; and
heating a portion of the endometrial lining of the uterus that is in thermal contact with the resistive element to thereby cause tissue necrosis.

21. The method according to claim 20, wherein the expandable element is a distendable bladder.

22. The method according to claim 21, wherein the expansion device is a device for injecting an inflation medium into the distendable bladder.

23. The method according to claim 22, wherein the device for injecting is a syringe.

24. A device for ablating an uterus comprising:
an introducer having a distal end and a proximal end and at least one channel therethrough;
a distendable bladder coupled to the distal end and being distendable within the body cavity from a substantially deflated state to an inflated state wherein it approximates an interior of at least a portion of said uterus;
an inflation device coupled to the proximal end and in fluid communication with the at least one channel and with an interior of the distendable bladder, wherein activation of the inflation device causes an inflation medium to flow through the at least one channel and into the distendable bladder to thereby inflate the distendable bladder; and
at least a first long, thin flexible element coupled to the distendable bladder in the region of the bladder in proximity to a first corneal area of the uterus when the bladder is positioned within the uterus, and at least a second long, thin flexible element coupled to the distendable bladder in the region of the bladder in proximity to a second corneal area of the uterus when the bladder is positioned within the uterus, the at least first and second flexible elements being coupled to the distendable bladder in a manner so as to direct expansion of the bladder into the corneal regions of the uterus.

25. The device according to claim 24, wherein the first and second flexible elements are arranged to form a pattern of substantially concentric circles, with the center of said substantially concentric circles being substantially aligned in the direction of the center of said respective corneal regions.
